(19) United States
(12) Patent Application Publication
(10) Pub. No.: US 2022/0199204 A1
(43) Pub. Date: Jun. 23, 2022

(54) ITERATIVE STATE DETECTION FOR MOLECULAR DYNAMICS DATA

(71) Applicant: International Business Machines Corporation, Armonk, NY (US)

(72) Inventors: Eun Kyung Lee, Bedford Corners, NY (US); Sara Kokkila Schumacher, Superior, CO (US); Nicolas Dupuis, New York, NY (US); Guojing Cong, Ossining, NY (US)

(21) Appl. No.: 17/126,421

(22) Filed: Dec. 18, 2020

(51) Int. Cl. G16C 20/50; G16C 20/30; G16C 10/00; G06F 30/20

(52) U.S. Cl. CPC G16C 20/50; G06F 30/20; G16C 10/00; G16C 20/30

(57) ABSTRACT

A method of finding an unknown molecular dynamics state includes receiving input molecular dynamics simulation data, determining a current layer of data from the input molecular dynamics simulation data, separating abnormal data from the current layer of data, extracting a targeted state using the abnormal data, and separating targeted state data from the current layer of data using the targeted state

### BACKGROUND

The present invention relates to methods of analyzing large data sets and more particularly to a method of identifying unknown molecular dynamic (MD) physical states and corresponding samples.

Large-scale MD simulations generate millions of frames of data, which precludes manual analysis.

### BRIEF SUMMARY

According to an embodiment of the present invention, a method for finding an unknown molecular dynamics state includes receiving input molecular dynamics simulation data, determining a current layer of data from the input molecular dynamics simulation data, separating abnormal data from the current layer of data, extracting a targeted state using the abnormal data, and separating targeted state data from the current layer of data using the targeted state extracted using the abnormal data.

According to some embodiments, a non-transitory computer readable medium comprising computer executable instructions which when executed by a computer system cause the computer to perform the method for finding an unknown molecular dynamics state comprises receiving input molecular dynamics simulation data, determining a current layer of data from the input molecular dynamics simulation data, separating abnormal data from the current layer of data, extracting a targeted state using the abnormal data, and separating targeted state data from the current layer of data using the targeted state.

According to at least one embodiment, A system configured to perform an iterative method of finding unknown molecular dynamics states and corresponding samples, the system comprising a communication interface configured to receive molecular dynamics data, the molecular dynamics data simulating movement of particles, a processor configured to determine a current layer of data from the molecular dynamics data, separate abnormal data from the current layer of data, extract a targeted state using the abnormal data, and separate targeted state data from the current layer of data using the targeted state extracted using the abnormal data, and a memory configured to store the targeted state and its data derived from the molecular dynamics data.

As used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

One or more embodiments of the invention or elements thereof can be implemented in the form of a computer program product including a computer readable storage medium with computer usable program code for performing the method steps indicated. Furthermore, one or more embodiments of the invention or elements thereof can be implemented in the form of a system (or apparatus) including a memory, and at least one processor that is coupled to the memory and operative to perform exemplary method steps. Yet further, in another aspect, one or more embodiments of the invention or elements thereof can be implemented in the form of means for carrying out one or more of the method steps described herein; the means can include (i) hardware module(s), (ii) software module(s) stored in a computer readable storage medium (or multiple such media) and implemented on a hardware processor, or (iii) a combination of (i) and (ii); any of (i)-(iii) implement the specific techniques set forth herein.

Techniques of the present invention can provide substantial beneficial technical effects. For example, one or more embodiments may provide for:

an iterative method of finding unknown molecular dynamics states and corresponding samples;

an anomaly detection module (ADM) that separates abnormal data from the total (nth-layer) data;

a state detection module (SDM) that identifies and extracts a targeted state using the abnormal data; and

a data separation module that separates targeted state data from the nth-layer data using the targeted state.

These and other features and advantages of the present invention will become apparent from the following detailed description of illustrative embodiments thereof, which is to be read in connection with the accompanying drawings.

### BRIEF DESCRIPTION OF THE SEVERAL VIEWS OF THE DRAWINGS

Preferred embodiments of the present invention will be described below in more detail, with reference to the accompanying drawings:

FIG. 1 is a diagram of system configured to perform an iterative method of finding unknown molecular dynamics state structures and corresponding samples according to at least one embodiment of the present invention;

FIG. 2 is a flow diagram of methods of finding unknown molecular dynamics state structures and corresponding samples according to at least one embodiment of the present invention;

FIG. 3 shows a histogram of a 1^{st} latent variable according to some embodiments of the present invention;

FIG. 4 illustrates of a state detection according to embodiments of the present invention;

FIG. 5 illustrates normal data separated according to embodiments of the present invention;

FIG. 6 illustrates abnormal data separated according to embodiments of the present invention; and

FIG. 7 is a block diagram depicting an exemplary computer system embodying an iterative method of finding unknown MD state structures and corresponding samples, according to an exemplary embodiment of the present invention.

### DETAILED DESCRIPTION

Molecular Dynamics (MD) describes a class of computer simulation methods for analyzing the physical movements of particles such as atoms or molecules. MD simulations are a tool for the exploration of, for example, the conformational energy landscape accessible to molecules or other particles, interactions between different molecules or particles, etc. Embodiments of the present invention are directed to an iterative method of finding unknown MD state structures and corresponding samples (e.g., data points corresponding to a particular/atom or group of particles/atoms). Embodiments of the present invention identify statistically meaningful states in the data, which may be rare. Investigating unknown state structures identified by MD data (trajectories/frames) analysis can lead to the identification of, for example, new drug targets.

Embodiments of the present invention are described in the context of unknown molecular dynamic structures. An example data set can be collected using classical molecular dynamics simulation campaigns. In a particular example, a data set can be collected using a massively parallel Multiscale Machine-Learned Modeling Infrastructure (MuMMI). This tool couples a macro scale model spanning micrometer length- and millisecond time-scales with a micro scale model of generated molecular dynamics simulations that are consistent with snapshots of the macro scale simulation. Embodiments of the present invention are not limited to the methods of data collection described herein.

The example dataset used herein for purposes of describing embodiments includes of over 116,000 coarse-grained Martini molecular dynamics simulations of various lipid membrane compositions and one or more wild-type GTP-loaded KRAS4b proteins, wherein GTP refers to the nucleotide guanosine triphosphate. Embodiments of the present invention enable processing of large data sets, e.g., on the order of hundreds of terabytes.

In the Martini model molecular dynamics approach, groups of atoms are represented as beads with defined physical parameters. The example dataset is a single KRAS4b protein molecular dynamics simulation subset, with every five MD time frames skipped, of the MuMMI generated data. Some embodiments of the present invention analyze the protein positions in the example dataset. Thus, according to some embodiments, each simulation data set is further simplified to only the protein Martini coarse grain beads, resulting in each simulation of 184 Martini beads (x,y,z coordinates in a periodic simulation box) and varying simulation lengths (resulting in different numbers of MD frames). Embodiments of the iterative method described here evaluate each MD frame.

It should be understood that embodiments of the present invention are described in the context of an example dataset, and that embodiments are not limited thereto. That is, embodiments are applicable to datasets for many-particle systems, including, molecules, proteins, gases, liquids, etc. Embodiments of the present invention can characterize a wide variety of molecular dynamics simulations and is generalizable beyond a single protein.

As the majority of molecular dynamics simulation data frames follow energetically stable patterns (e.g., shape, relative location of the coarse grain beads, etc.), embodiments of the present invention identify unknown states by searching for abnormal data.

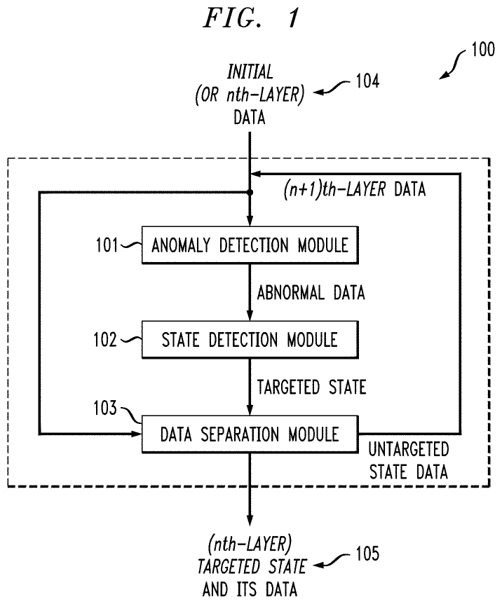

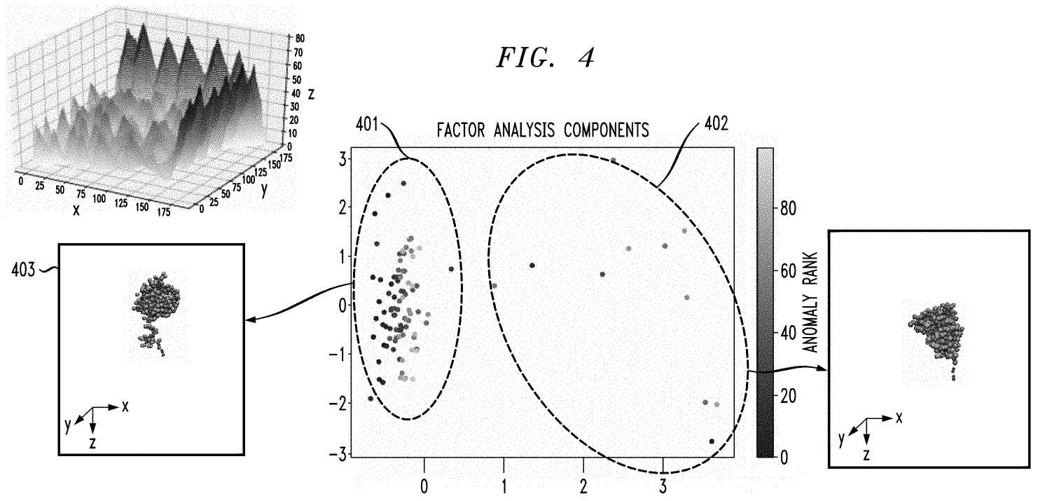

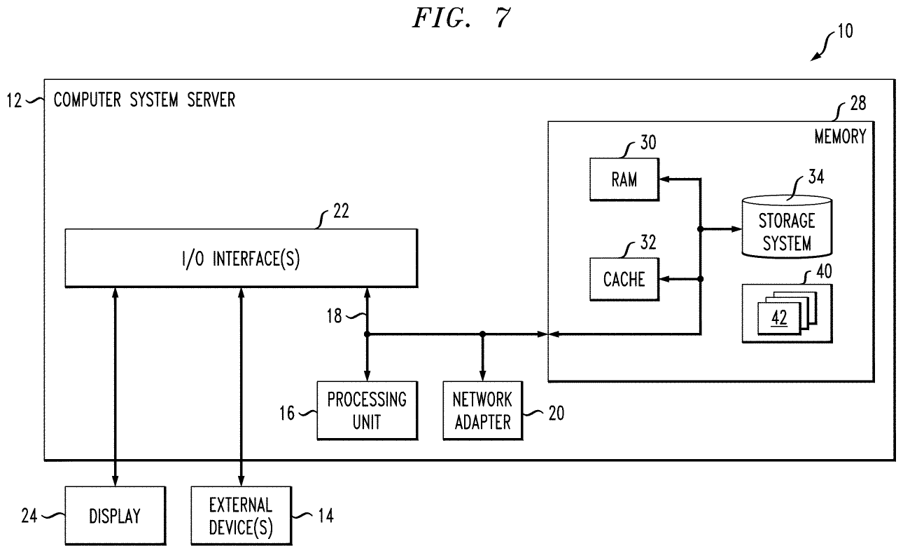

Referring to FIG. 1, a system 100 configured to perform an iterative method of finding unknown MD states and corresponding samples according to at least one embodiment of the present invention comprises an anomaly detection module (ADM) 101 separates abnormal data from the total (n^{th} layer) data 104. The layers of data are defined for the iterative method. More particularly, each layer is a defined set of the MD simulation data, which has a statistical and/or structural meaning for a researcher or user. By way of example, the RAS protein with an elongated farnesyl group 403 in FIG. 4 is one example of a layer, where one targeted state defines one layer of a data set in a 1:1 mapping. A state detection module (SDM) 102 identifies and extracts a specific state using the abnormal data separated in ADM. The specific state is a targeted state. A data separation module 103 separates the targeted state data from the n^{th} layer data using the targeted state detected in SDM. The system iteratively performs a method (see FIG. 6) processing data in each successive layer of data (i.e., processing a (n+1)^{th} layer) using untargeted data in the n^{th} layer, outputting a targeted state and its data 105 for each iteration. According to at least one embodiment, the system stops iterating when the untargeted state data meets a stopping criteria.

According to some embodiments, a system 12 configured to perform an iterative method of finding unknown molecular dynamics states and corresponding samples includes a communication interface (e.g., see 22, FIG. 7) configured to receive molecular dynamics data (e.g., from a storage device), the molecular dynamics data simulating the movement of particles, at least one processor 16, configured to receive molecular dynamics data, determine a current layer of data from the molecular dynamics data, separate abnormal data from the current layer of data, extract a targeted state using the abnormal data, and separate the targeted state data from the current layer of data using the targeted state extracted using the abnormal data, and a memory 28 configured to store the targeted state and its data derived from the molecular dynamics data.

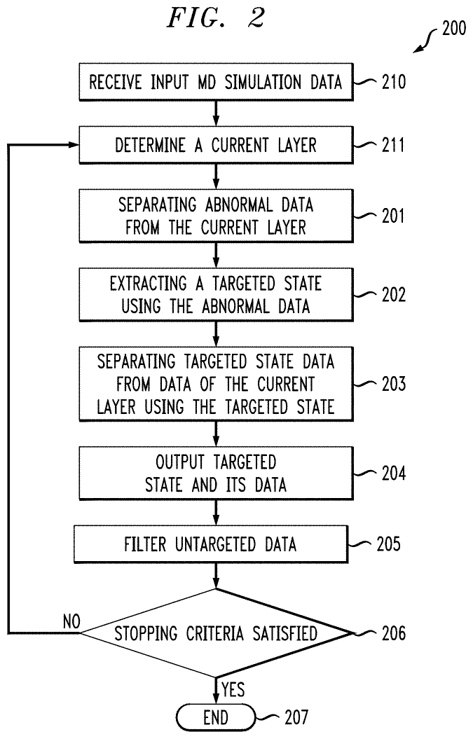

According to some embodiments and referring to FIG. 2, a method 200 for finding unknown molecular dynamics states comprises separating abnormal data from a current layer of data 201, extracting a targeted state from the abnormal data 202, and separating targeted state data from the n^{th} layer data using the extracted targeted state 203. Method 200 iterates through n layers of data, processing the (n+1)^{th} layer using untargeted data in the n^{th} layer. That is, a current layer is processed using the untargeted data from the previous layer. At each iteration the method outputs the targeted state and its data at 204, determines whether there are additional layers 205-206, and if so increments the current layer 211 (e.g., current layer n=n+1) before starting a next iteration, and if not, ends the simulation 207.

According to some embodiments, the extraction of the targeted state from the abnormal data 202 includes sampling the abnormal data to determining targeted samples, and inferring (e.g., by statistical inference) the targeted state from the targeted samples. Thus, the targeted state is determined from the abnormal data. The extracted targeted samples are treated as statistically/structurally meaningful. The extraction of the targeted samples can address noise in the abnormal data, e.g., by systematic sampling or cluster sampling. Other methods of sampling are possible. Exemplary methods for finding the targeted state are described herein in connection with state detection module (SDM) 102.

According to at least one embodiment, the extraction of the targeted samples from the abnormal data at 202 is optional. For example, if the abnormal data would be the same as the targeted samples, then the sampling of the abnormal data can be skipped.

At block 210 the ADM receives input molecular dynamics simulation data. At block 211, the ADM can treat the entirety of the input molecular dynamics simulation data as the current layer of data, or can sample the input molecular dynamics simulation data to reduce a size of the data to be processed.

It should be understood that embodiments of the present invention can be applied as an improved method of visualizing MD data, wherein the output of targeted state and its data 204 includes a visualization (see for example 403) of the data (a non-conventional method for visualizing MD data extracted according to one or more embodiments). As described above, it should be appreciated that some embodiments enable processing of large-scale data, not previously possible, for the identification of unknown states.

It should be understood that embodiments of the present invention are described in the context of data points, and that the data points correspond to beads in a protein MD simulation. It should further be understood that embodiments of the present invention are applicable to data points corresponding to any data characterized as a particle in a many-particle system. Accordingly, embodiments of the present invention are not limited to data points corresponding to beads in a protein MD simulation.

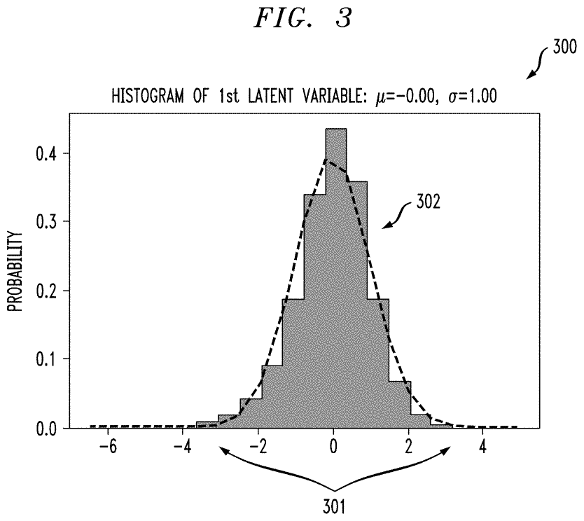

Referring to FIG. 3 and the anomaly detection module (ADM) 101, the ADM separates abnormal data 301 from normal data points 302 (see also 101), wherein abnormal and normal can be defined statistically. Graph 300 shows an anomaly detection using an autoencoder and its latent variables, μ (mean value) and σ (standard deviation). (It should be understood that an autoencoder is an unsupervised learning technique that leverages neural networks for the task of representation learning.) The ADM calculates an absolute value of the z-scores (probability) of the latent (inferred) variables and ranks them to find the abnormal data points. It should be understood that a threshold to separate normal and abnormal data can be predefined, for example, the threshold can be defined as the absolute value of three standard deviations (i.e., abs(3σ)), substantially as illustrated by 301. According to some embodiments, the threshold is set by a user.

Referring to FIG. 4 and the state detection module (SDM) 102, the SDM identifies and extracts a specific state (the targeted state) using the abnormal data separated by the ADM (see also 202). According to some embodiments, the SDM extracts the targeted samples from abnormal data, the targeted samples exemplifying the targeted state. According to at least one embodiment, the SDM utilizes a clustering algorithm to find the targeted state 401 based on the identification of the targeted samples among the abnormal data. According to some embodiments, the SDM can use a factor analysis to find the targeted state (e.g., Ras proteins with an elongated farnesyl group, which are used in cancer research, see image 403). Example factor analysis methods include Principal Component Analysis (PCA), Support Vector Machine (SVM), Linear Discriminant Analysis (LDA).

According to some embodiments, the untargeted data 402 is identified as data not statistically relevant to the targeted data in the current iteration (i^{th} iteration). The untargeted data of the abnormal samples from block 205 is reused as input for next iteration (i+1^{th} iteration) (see block 211). Accordingly, layers are determined iteratively according to the method of FIG. 2.

According to some embodiments and referring to the data separation module (DSM) 103, the DSM separates the targeted state data using the targeted state detected by the SDM (see also 203). According to some embodiments, a clustering algorithm (e.g., a factor analysis) can be used to separate the targeted state data from abnormal data. For example, the targeted data can include data within some threshold measure (e.g., distance) from a center of a cluster (see FIG. 3) (for example, targeted data=data (∥data−center_cluster∥<threshold).

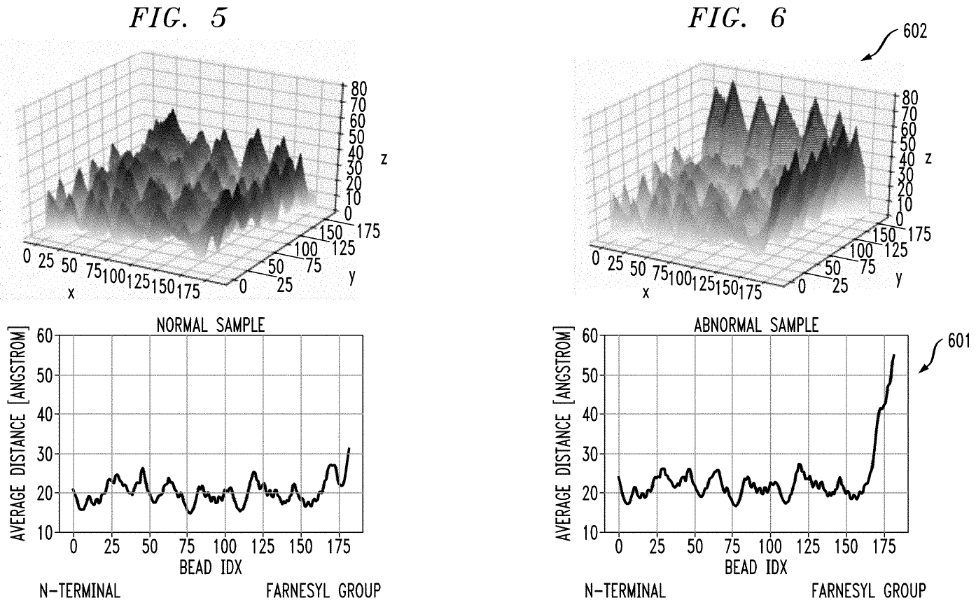

Before discussion FIG. 5 and FIG. 6, it should be understood that in the particular case of protein analysis, a bead (see x-axis of graph 601) represents a group of atoms/molecules in a given simulation. The example RAS protein used in FIG. 5 and FIG. 6 contains 184 beads from N-terminal through farnesyl group (the start of a protein is the N-terminal, the end of a protein is a C-terminal, and in the example data set a farnesyl group is attached to the protein C-terminal).

According to some embodiments and referring to FIG. 5 and FIG. 6, the DSM 103 uses a threshold-based method for data separation. For example, a threshold can be set on a mean distance matrix data to detect Ras proteins with an elongated farnesyl group (see FIG. 6). In n^{th} layer data, targeted data (with an elongated farnesyl group) is separated. Since the average distance or length for the farnesyl group is high for abnormal samples, it is feasible to separate abnormal samples (see FIG. 6) from the normal samples (see FIG. 5). It should be understood that elongated farnesyl group data of a distribution is the portion of the distribution having many occurrences far from the N-terminal or central part of the distribution (see graphs 601 and 602). The parameters of the data separation (i.e., what is considered elongated farnesyl group data) can be predetermined (e.g., the most frequently occurring 20% of items represent less than 50% of occurrences) or set by a user.

According to some embodiments, in the (n+1)^{th} iteration, the ADM (block 201 of FIG. 2) and the DSM (block 203, FIG. 2) use the n^{th} layer untargeted data (see for example, data 402 in FIG. 4) separated by the DSM during the n^{th} iteration (i.e., the prior iteration).

According to some embodiments, a portion of the untargeted data can be filtered out. For example, a portion of the untargeted data can be identified as not statistically relevant or noisy and filtered at block 205 of FIG. 2.

According to some embodiments, at block 206 the ADM can stop the method 207 based on a stopping criteria, such as when the untargeted state data reaches a certain data count (i.e., number of samples). For example, at block 206 the ADM can end a simulation 207 when the untargeted state data exceeds a threshold of 90% of the total data counts of the input data (the data input at 210). Alternatively, the method proceeds to blocks 201-202 where the ADM separates abnormal data from a current layer of data and extracts a targeted state using the abnormal data.

According to some embodiments, input molecular dynamics simulation data 210 to the ADM can be subsampled at block 211. For example, in a case where the method of the ADM is known to be computationally expensive with respect to the number of input samples. Further, for the n+1 iteration, the current layer at 211 is the untargeted data from n^{th} iteration determined at block 205.

Embodiments of the present invention are applicable to deep learning and dimensionality reduction approaches to detecting rare events and anomalies in MD simulation data.

Recapitulation:

According to some embodiments, a method for finding unknown molecular dynamics state includes receiving molecular dynamics simulation data 210, determining a current layer of data from the input molecular dynamics simulation data 211, separating abnormal data from the current layer of data 201, extracting a targeted state using the abnormal data 202, and separating targeted state data from the current layer of data using the targeted state extracted using the abnormal data 203.

According to at least one embodiment, a system 12 configured to perform an iterative method of finding unknown molecular dynamics states and corresponding samples, the system comprising a communication interface 22 configured to receive molecular dynamics data, the molecular dynamics data simulating movement of particles, a processor 16 configured to determine a current layer of data from the molecular dynamics data, separate abnormal data from the current layer of data, extract a targeted state using the abnormal data, and separate targeted state data from the current layer of data using the targeted state extracted using the abnormal data, and a memory 28 configured to store the targeted state and its data derived from the molecular dynamics data.

The methodologies of embodiments of the disclosure may be particularly well-suited for use in an electronic device or alternative system. Accordingly, embodiments of the present invention may take the form of an entirely hardware embodiment or an embodiment combining software and hardware aspects that may all generally be referred to herein as a “processor,” “circuit,” “module” or “system.”

Furthermore, it should be noted that any of the methods described herein can include an additional step of providing a computer system implementing an improved gaze tracking method (re)configurable for a multi-display environment. Further, a computer program product can include a tangible computer-readable recordable storage medium with code adapted to be executed to carry out one or more method steps described herein, including the provision of the system with the distinct software modules.

One or more embodiments of the invention, or elements thereof, can be implemented in the form of an apparatus including a memory and at least one processor that is coupled to the memory and operative to perform exemplary method steps. FIG. 7 depicts a computer system that may be useful in implementing one or more aspects and/or elements of the invention, also representative of a cloud computing node according to an embodiment of the present invention. Referring now to FIG. 7, cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 7, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, and external disk drive arrays, RAID systems, tape drives, and data archival storage systems, etc.

Thus, one or more embodiments can make use of software running on a general purpose computer or workstation. With reference to FIG. 7, such an implementation might employ, for example, a processor 16, a memory 28, and an input/output interface 22 to a display 24 and external device(s) 14 such as a keyboard, a pointing device, or the like. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory) 30, ROM (read only memory), a fixed memory device (for example, hard drive 34), a removable memory device (for example, diskette), a flash memory and the like. In addition, the phrase “input/output interface” as used herein, is intended to contemplate an interface to, for example, one or more mechanisms for inputting data to the processing unit (for example, mouse), and one or more mechanisms for providing results associated with the processing unit (for example, printer). The processor 16, memory 28, and input/output interface 22 can be interconnected, for example, via bus 18 as part of a data processing unit 12. Suitable interconnections, for example via bus 18, can also be provided to a network interface 20, such as a network card, which can be provided to interface with a computer network, and to a media interface, such as a diskette or CD-ROM drive, which can be provided to interface with suitable media.

Accordingly, computer software including instructions or code for performing the methodologies of the invention, as described herein, may be stored in one or more of the associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 16 coupled directly or indirectly to memory elements 28 through a system bus 18. The memory elements can include local memory employed during actual implementation of the program code, bulk storage, and cache memories 32 which provide temporary storage of at least some program code in order to reduce the number of times code must be retrieved from bulk storage during implementation.

Input/output or I/O devices (including but not limited to keyboards, displays, pointing devices, and the like) can be coupled to the system either directly or through intervening I/O controllers.

Network adapters 20 may also be coupled to the system to enable the data processing system to become coupled to other data processing systems or remote printers or storage devices through intervening private or public networks. Modems, cable modem and Ethernet cards are just a few of the currently available types of network adapters.

As used herein, including the claims, a “server” includes a physical data processing system (for example, system 12 as shown in FIG. 7) running a server program. It will be understood that such a physical server may or may not include a display and keyboard.

It should be noted that any of the methods described herein can include an additional step of providing a system comprising distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as 16. Further, a computer program product can include a computer-readable storage medium with code adapted to be implemented to carry out one or more method steps described herein, including the provision of the system with the distinct software modules.

One example of user interface that could be employed in some cases is hypertext markup language (HTML) code served out by a server or the like, to a browser of a computing device of a user. The HTML is parsed by the browser on the user's computing device to create a graphical user interface (GUI).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of finding an unknown molecular dynamics state comprising:
receiving input molecular dynamics simulation data;
determining a current layer of data from the input molecular dynamics simulation data;
separating abnormal data from the current layer of data;
extracting a targeted state using the abnormal data; and
separating targeted state data from the current layer of data using the targeted state.

2. The method of claim 1, wherein the method iterates through a plurality of layers of data, wherein at each iteration the method processes a next layer comprising untargeted data from a prior layer.

3. The method of claim 2, wherein the input molecular dynamics simulation data is the current layer of data for a first iteration and the targeted state defines the current layer of data for a subsequent iteration.

4. The method of claim 2, wherein the method outputs the targeted state and the targeted state data from each iteration.

5. The method of claim 2, wherein the method ends upon determining that a ratio of untargeted data to total data is greater than a threshold.

6. The method of claim 1, wherein determining the current layer of data from the input molecular dynamics simulation data comprises sampling the input molecular dynamics simulation data to reduce a size of the current layer of data in a first iteration.

7. The method of claim 1, where the abnormal data is separated from the current layer of data by an autoencoder.

8. The method of claim 1, wherein the extraction of the targeted state further comprises a first clustering finding targeted samples among abnormal samples separated from the current layer of data, the target samples exemplifying the targeted state.

9. The method of claim 1, wherein separating the targeted state data from the current layer of data comprises a second clustering, the second clustering separating the targeted state data from the current layer of data using the targeted state.

10. The method of claim 9, wherein the second clustering uses a measure of distance from a center of a cluster of the current layer of data and a threshold for the measure of distance.

11. A non-transitory computer readable medium comprising computer executable instructions which when executed by a computer system cause the computer to perform the method for finding an unknown molecular dynamics state comprising:
receiving input molecular dynamics simulation data;
determining a current layer of data from the input molecular dynamics simulation data;
separating abnormal data from the current layer of data;
extracting a targeted state using the abnormal data; and
separating targeted state data from the current layer of data using the targeted state.

12. The computer readable medium of claim 11, wherein the method iterates through a plurality of layers of data, wherein at each iteration the method processes a next layer comprising untargeted data from a prior layer.

13. The computer readable medium of claim 12, wherein the input molecular dynamics simulation data is the current layer of data for a first iteration and the targeted state defines the current layer of data for a subsequent iteration.

14. The computer readable medium of claim 12, wherein the method outputs the targeted state and the targeted state data from each iteration, and wherein the method ends upon determining that a ratio of untargeted data to total data is greater than a threshold.

15. The computer readable medium of claim 11, where the abnormal data is separated from the current layer of data by an autoencoder.

16. The computer readable medium of claim 11, wherein the extraction of the targeted state further comprises a first clustering finding targeted samples among abnormal samples separated from the current layer of data, the target samples exemplifying the targeted state.

17. The computer readable medium of claim 11, wherein separating the targeted state data from the current layer of data comprises a second clustering, the second clustering separating the targeted state data from the current layer of data using the targeted state.

18. The computer readable medium of claim 19, wherein the second clustering uses a measure of distance from a center of a cluster of the current layer of data and a threshold for the measure of distance.

19. A system configured to perform an iterative method of finding unknown molecular dynamics states and corresponding samples, the system comprising:
a communication interface configured to receive molecular dynamics data, the molecular dynamics data simulating movement of particles;
a processor configured to determine a current layer of data from the molecular dynamics data, separate abnormal data from the current layer of data, extract a targeted state using the abnormal data, and separate targeted state data from the current layer of data using the targeted state extracted using the abnormal data; and
a memory configured to store the targeted state and its data derived from the molecular dynamics data.

20. The system of claim 19, further comprising a display controlled by the processor to display the targeted state data.
